(19) United States
(12) Patent Application Publication
(10) Pub. No.: US 2025/0308692 A1
(43) Pub. Date: Oct. 2, 2025

(54) ANOMALY DETECTION METHOD AND SYSTEM BASED ON PU CONTRASTIVE LEARNING WITHIN MULTIMODAL PROTOTYPE NETWORK

(71) Applicant: Hefei University of Technology, Hefei (CN)

(72) Inventors: Richang HONG, Hefei (CN); Yuqi CHU, Hefei (CN); Yanrong GUO, Hefei (CN); Shijie HAO, Hefei (CN)

(73) Assignee: Hefei University of Technology, Hefei (CN)

(21) Appl. No.: 18/820,279

(22) Filed: Aug. 30, 2024

(30) Foreign Application Priority Data
Mar. 27, 2024 (CN) 202410354820.4

(51) Int. Cl. G16H 50/20; G06N 20/00

(52) U.S. Cl. CPC G16H 50/20; G06N 20/00

(57) ABSTRACT

An anomaly detection method based on PU contrastive learning within a multimodal prototype network that employs dilated convolutional networks and Bert models to form a multimodal data (EEG and text) feature extraction and fusion network. Through a multimodal feature enhanced prototype network, clustering is performed, but the results are biased due to the lack of labeled negative samples. Finally, a positive unlabeled learning method that integrates contrastive learning is used to estimate the unbiased risk of the biased clustering results, correct the deviation, and accurately identify the positive and negative samples. By analyzing a limited number of multimodal positive samples and a large amount of unlabeled data, the anomaly detection method can accurately classify positive samples and negative samples without the need for expensive manual labeling costs. It also adopts a self-supervised learning framework, integrating PU learning into contrastive learning to correct the classification deviation.

### CROSS REFERENCE TO THE RELATED APPLICATIONS

This application is based upon and claims priority to Chinese Patent Application No. 202410354820.4, filed on Mar. 27, 2024, the entire contents of which are incorporated herein by reference.

### TECHNICAL FIELD

The present disclosure relates to the technical field of artificial intelligence for medical, in particular to an anomaly detection method and system based on positive unlabeled (PU) contrastive learning within a multimodal prototype network.

### BACKGROUND

In today's medical diagnostic field, it is becoming more and more important to accurately and quickly diagnose a patient's disease. Traditional medical diagnosis methods mainly rely on the experience and knowledge of doctors, which are time-consuming and inefficient, unable to deal with a large number of patient data, and lack of automation and intelligence. With its excellent performance in image recognition, natural language processing and in-depth learning, medical artificial intelligence technology is gradually becoming an important tool to assist doctors in diagnosis and solve the above problems. However, the existing medical intelligent detection methods are usually single modal detection, which has the problems of limited detection accuracy and a large number of labeled samples, and the labeling cost is high. In order to improve the accuracy of detection, multimodal data can be used for analysis, but in the medical diagnostic scene, it is extremely difficult and costly to obtain the labeled multimodal (electroencephalography (EEG), text) positive sample data and negative sample data, usually, the obtained multimodal data are partial labeled positive samples and a large number of unlabeled samples. This data imbalance makes it particularly difficult to train an effective classifier, and the deviation is large.

In order to solve the above problems, most of the existing multimodal anomaly detection methods use data enhancement techniques to balance the positive and negative samples, such as flipping, splitting or adding noise to artificially increase the number of positive samples, but this way can not fully capture the intrinsic correlation of data, and may introduce additional noise. In addition, some advanced methods use semi-supervised learning to learn from unlabeled data, attempting to extract useful information from a large number of unlabeled samples. However, it usually requires careful design of loss functions and training strategies, facing problems such as difficult model convergence and easy collapse.

Therefore, it is an urgent problem for those skilled in the art to provide an anomaly detection method based on PU contrastive learning within a multimodal prototype network to achieve accurate and effective binary classification in the context of imbalanced multimodal data and limited resources.

### SUMMARY

In view of this, the present disclosure provides an anomaly detection method and system based on PU contrastive learning within a multimodal prototype network, which uses a dilated convolutional network and a Bidirectional Encoder Representations from Transformers (BERT) model to establish a multimodal data (EEG, text) feature extraction and fusion network, and then uses the multimodal feature enhanced prototype network to cluster the fused features. Due to the lack of labeled negative samples and the imbalance of data, the clustering results are biased. In order to solve this problem, the self-supervised contrast learning strategy combined with PU learning is used to estimate the unbiased risk of the above clustering results and determine the category of the samples, aiming at accurately identifying the positive samples and the negative samples from the unlabeled samples.

In order to achieve the above effects, the present disclosure adopts the following technical solutions.

On one hand, an anomaly detection method based on PU contrastive learning within a multimodal prototype network is disclosed by the present disclosure, which includes the following steps:
acquiring multimodal data, wherein the multimodal data includes EEG modal data and text modal data;
constructing and training an unbiased classification model, wherein the unbiased classification model includes a feature extraction and fusion network, a multimodal feature enhanced prototype network and a PU contrastive learning network; the feature extraction and fusion network generates a fusion feature according to the multimodal data, the fusion feature is clustered by the multimodal feature enhanced prototype network, and a certain deviation exists in the clustering result; and finally, carry out unbiased risk estimation on that biased cluster result by using the PU contrastive learning network, correct the deviation cause by the lack of labeled negative samples and data imbalance, and realizing the classification of the multimodal data; and
inputting the multimodal data into a trained unbiased classification model, and outputting the category to which the multimodal data belongs.

Preferably, the acquiring the multimodal data includes preprocessing the multimodal data;

dividing the multimodal data into a partial labeled positive sample X_{P}, X_{P}={x₁, x₂, . . . , xₙ_{ₚ}} and an unlabeled sample X_{U}, X_{U}={x₁, x₂, . . . , xₙ_{ᵤ}}, wherein the label of the partial labeled positive sample X_{P} is Y=+1, and the unlabeled sample X_{U} has no label.

Preferably, the feature extraction and fusion network includes a dilated convolutional network, a BERT model, and a multi-head self-attention mechanism.

The dilated convolutional network and the BERT model are respectively configured to extract the features of the EEG modal data and the text modal data, and the multi-head self-attention mechanism is configured to fuse the features of the multimodal data to generate the fusion features.

Preferably, the multimodal feature enhanced prototype network calculates k-class prototypes cₖ by fusing features, cₖ={c_{P}, c_{U}}, wherein c_{P} is the prototype of the partial labeled positive sample X_{P}, i.e., the positive class prototype, c_{P}∈R^{d}; c_{U} is a prototype of the unlabeled sample X_{U}, i.e., the unlabeled prototype c_{U}∈R^{d}, but the unlabeled data includes positive samples and negative samples, so the unlabeled prototype cy has a certain deviation;

Calculate the Euclidean distance between each sample xᵢ and each prototype cₖ in the embedding space to obtain the probability distribution p_{ϕ}(y=k|xᵢ) of a binary classification.

Preferably, the loss function of the multimodal feature enhanced prototype network is

$${L}_{m p n} = \frac{1}{\vert X \vert} [ {‖ O ( {x}_{i} ) - {c}_{k} ‖}_{2}^{2} + log {\sum }_{k} exp ( - {‖ O ( {x}_{i} ) - {c}_{k} ‖}_{2}^{2} ) ] ;$$

wherein, X is the data sample, and O(xᵢ) is the fusion feature of the sample xᵢ.

Preferably, the PU contrastive learning network merges the fusion feature with the prototype cₖ to obtain a sample pair Zᵢ; and an unbiased risk estimation function, i.e., a PU contrastive learning loss function, is constructed based on the sample pair Zᵢ.

Preferably, merge the fusion feature with the prototype cₖ to obtain a sample pair Zᵢ includes:

The positive sample pair Z_{P}ⁱ, Z_{P}ⁱ={z_{P}¹, z_{P}², . . . , z_{P}ⁿ^{ₚ}} is calculated by the fusion feature O_{P} of the partial labeled positive sample X_{P} and the positive class prototype, and the sample pair Z_{U}^{j}, Z_{U}^{j}={z_{U}¹, z_{U}², . . . , z_{U}ⁿ^{ᵤ}} is calculated by the fusion feature O_{U} of the unlabeled sample X_{U} and the unlabeled prototype c_{U}.

Preferably, the unbiased risk estimation function, that is, the PU contrastive learning loss function, is

$${L}_{c p u}^{( i , j )} = - log \frac{exp ( {Z}_{P}^{i} \cdot {π}_{P} {Z}_{U}^{j} )}{\begin{matrix}{\sum }_{i ∈ \{ 1 , 2 , … {n}_{p} \} , j ∈ \{ 1 , 2 , … {n}_{u} \}} ( exp ( {Z}_{P}^{i} \cdot {π}_{P} {Z}_{U}^{j} ) + \\ exp ( max ( 1 {e}^{- 5} , {Z}_{P}^{i} \cdot ( 1 - {π}_{P} ) {Z}_{U}^{{j}^{′}} ) ) )\end{matrix}} ;$$

wherein π_{P} is the prior probability of the positive sample.

Preferably, the inputting the multimodal data into a trained unbiased classification model, and outputting the category to which the multimodal data belongs includes:
inputting the multimodal data into the feature extraction and fusion network to generate a fusion feature Oᵢ;
calculating the Euclidean distance between the fusion feature Oᵢ and each prototype cₖ in the multimodal feature enhanced prototype network in an embedding space to obtain a probability distribution p_{ϕ} of a sample classification; and
deciding the category to which the multimodal data belongs according to the probability distribution p_{ϕ}.

On the other hand, an anomaly detection system based on PU contrastive learning within a multimodal prototype network is disclosed by the present disclosure, which is used to implement the aforementioned the anomaly detection system based on PU contrastive learning within a multimodal prototype network, including:
a data acquisition module, which is configured to acquire multimodal data, wherein the multimodal data includes EEG modal data and text modal data;
a model building module, which is configured to construct and train an unbiased classification model; the unbiased classification model includes a feature extraction and fusion network, a multimodal feature enhanced prototype network and a PU contrastive learning network; the feature extraction and fusion network generates a fusion feature according to the multimodal data, the fusion feature is clustered by multimodal feature enhanced prototype network to obtain a biased clustering result; and finally, carry out unbiased risk estimation on that biased cluster center by using the PU contrastive learning network, correcting the deviation cause by the lack of labeled negative samples and data imbalance, and realizing the classification of the multimodal data; and
a detection and output module, which is configured to input the multimodal data into a trained unbiased classification model, and output the category to which the multimodal data belongs.

According to the above technical solutions, compared with the prior art, the present disclosure discloses an anomaly detection method and system based on PU contrastive learning within a multimodal prototype network, which aim at the classification task of multimodal unbalanced data, are particularly suitable for scenes with scarce positive samples and high labeling cost in the medical field, and can accurately distinguish the positive samples from the negative samples by analyzing limited multimodal positive samples and a large amount of unlabeled data. On the other hand, in order to solve the problem of deviation in the classification of unlabeled samples, contrastive learning is fused into PU learning, and through a contrastive learning algorithm, the feature representation in the multimodal data can be independently mined, the low-dimensional feature representation of the multimodal data for anomaly detection is obtained through learning, and high quality features are provided for a downstream multimodal classification task.

### BRIEF DESCRIPTION OF THE DRAWINGS

In order to more clearly illustrate the embodiments of the present disclosure or technical solutions in the related art, the accompanying drawings used in the embodiments or the related art will now be described briefly. It is obvious that the drawings in the following description are only the embodiment of the disclosure, and that those skilled in the art can obtain other drawings from these drawings without any creative efforts.

FIG. 1 is a flowchart of an anomaly detection method and system based on PU contrastive learning within a multimodal prototype network according to an embodiment of the present disclosure; and

FIG. 2 is a system architecture diagram of an anomaly detection method and system based on PU contrastive learning within a multimodal prototype network according to an embodiment of the present disclosure.

### DETAILED DESCRIPTION OF THE EMBODIMENTS

In the following, the technical solutions in the embodiments of the present disclosure will be clearly and completely described with reference to the drawings in the embodiments of the present disclosure. Obviously, the described embodiments are only a part of the embodiments of the present disclosure, but not all the embodiments thereof. Based on the embodiments of the present disclosure, all other embodiments obtained by those skilled in the art without any creative efforts shall fall within the scope of the present disclosure.

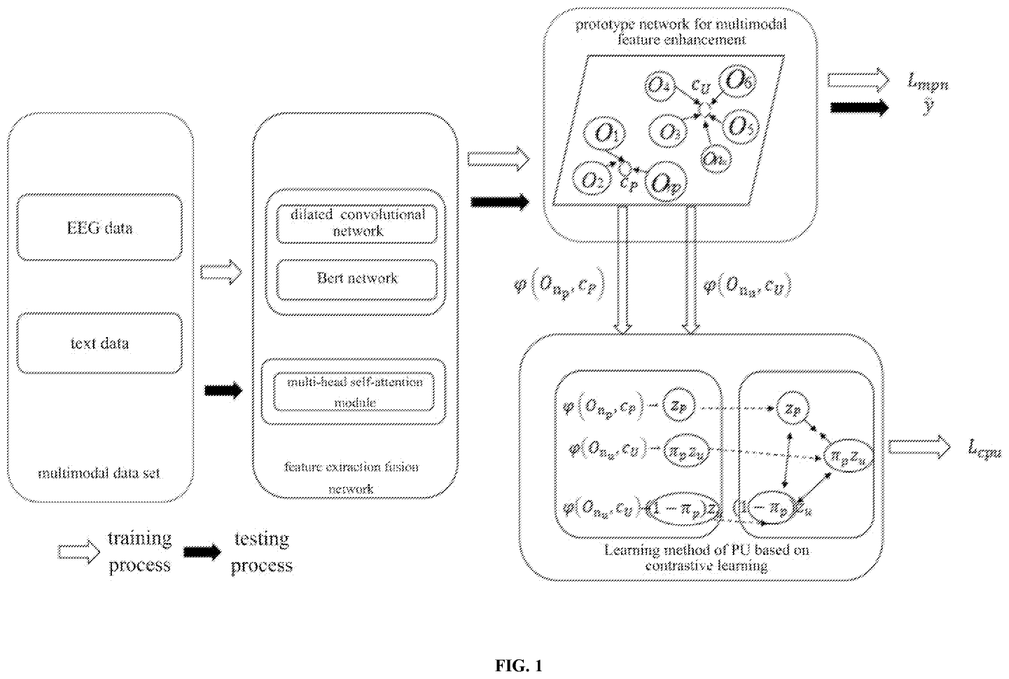

On one hand, an anomaly detection method based on PU contrastive learning within a multimodal prototype network is disclosed by the present disclosure, as shown in the FIG. 1, including the following steps.

S1, multimodal data is acquired, wherein the multimodal data includes EEG modal data and text modal data.

Wherein, acquiring the multimodal data includes preprocessing the multimodal data.

Assume that X and Y are data samples and labels, πₚ=p(Y=+1) and πₙ=p(Y=−1) are prior probabilities for positive samples and negative samples, and πₚ is known for positive samples. Multimodal data is divided into labeled positive samples X_{P}, X_{P}={x₁, x₂, . . . , xₙ_{ₚ}} and unlabeled samples X_{U}, X_{U}={x₁, x₂, . . . , xₙ_{ᵤ}}; the label of labeled positive sample X_{P} is Y=+1, and the label of unlabeled sample X_{U} is missing.

S2, the unbiased classification model is constructed and trained, wherein the unbiased classification model includes a feature extraction and fusion network, a multimodal feature enhanced prototype network and a PU contrastive learning network.

The feature extraction and fusion network generates a fusion feature according to the multimodal data, the fusion feature is clustered by multimodal feature enhanced prototype network; and finally, the imbalanced multimodal data features are classified by the PU contrastive learning network.

After training the above feature extraction and fusion network, multimodal feature enhanced prototype network, and PU contrastive learning network with partial annotated multimodal positive sample data and a large number of unlabeled multimodal samples, an unbiased classification model for practical inference testing can be obtained. The training process is shown by the white arrow in FIG. 1.

Specifically, in order to construct a more comprehensive and representative multimodal fusion feature vector, the embodiment of the present disclosure constructs a multimodal feature extraction and fusion network including a dilated convolutional network, a BERT model, and a multi-head self-attention mechanism.

Specifically, the dilated convolutional network is configured for feature extraction of EEG modal data to obtain a representation E={e₁, e₂, . . . , e_{N}} representing the EEG features of the sample, the BERT model is configured for feature extraction of text modal data to obtain a representation S={s₁, s₂, . . . , s_{N}} representing the text features of the sample, and the multi-head self-attention mechanism is configured for efficiently fusing the features of these two modalities to obtain the features O={o₁, o₂, . . . , o_{N}} of the fusion of EEG information and text information are obtained.

The architecture of the dilated convolutional network is composed of a linear projection layer, multiple dilated convolutional networks, and an output layer. The linear projection layer is a fully connected layer that maps the EEG data from its original feature dimensions (such as 3, 64, or 128) to 64 hidden channels. The dilated convolutional network is composed of four hidden blocks, and each hidden block is composed of a RELU layer, an dilated convolution layer, a RELU layer and an dilated convolution layer. Wherein, the number of dilated convolution channels per hidden block is 64, the size of the convolution kernel is 3, and the extension rate of the dilated convolution at the i-th layer is set to 21. The four hidden blocks are connected in series through the residual connection, and finally output through an output layer with a channel size of 256. For the pre-trained Bert model of the text modality, the dimension of its output feature vector is set to 256.

In the feature fusion stage, the EEG feature representation E and the text feature representation S are first merged through a splicing operation to generate a joint feature vector M=[m₁, m₂, . . . , m_{N}]∈R^{N×d} with a dimension of 512, and then three groups of trainable parameter matrices F₁^{h}, F₂^{h}, F₃^{h} are calculated for each head h due to the adoption of a multi-head (8 in total) self-attention mechanism:

F₁^{h}=MW₁, F₂^{h}=MW₂, F₃^{h}=MW₃
Wherein, W₁, W₂, W₃∈R^{d×d}^{ₕ}, dₕ is the feature dimension corresponding to each head h, and then the attention score Aᵢ of each head is calculated:

$${A}_{i} = softmax ( \frac{{F}_{1}^{i} \cdot {( {F}_{2}^{i} )}^{T}}{\sqrt{{d}_{h}}} ) {F}_{3}^{i}$$

Merge the 8 Aᵢ obtained through a splicing operation and perform linear transformation with the matrix W₄∈R^{h×d}^{ₕ}^{×d} to obtain the final fused features O:

O=[A₁∥A₂∥ . . . ∥Aₖ]W₄
Wherein, ∥ is the splicing operation.

In this embodiment, the fusion feature O_{P} of the labeled positive sample X_{P}, the fusion feature O_{U} of the unlabeled sample X_{U}, and the fusion feature Oᵢ obtained by training are respectively calculated according to the method for obtaining the fusion features O.

Through the weighting and optimization of the multi-head self-attention mechanism, the final fusion features O not only integrate the rich information from EEG data and text data, but also have higher representativeness and comprehensiveness. This mechanism enhances the ability of the model to capture the complex associations between different modalities more accurately, resulting in a more comprehensive and representative feature representation.

Further, in order to achieve accurate clustering in the feature embedding space, the present disclosure adopts a multimodal fusion feature enhanced prototype network, aiming to obtain a more discriminative prototype using multimodal features fused with EEG data and text data.

Because the task of the present disclosure is a binary classification task under the condition that only a part of positive sample labels and a large number of unlabeled samples exist, aiming at the problems of data imbalance and lack of labeled negative samples, in order to obtain a classification result with high accuracy, the key is to learn a prototype with discrimination. This not only allows for better retention of category-related information, at the same time, it can also reduce the deviation caused by the negative samples which are similar to the positive samples in the unlabeled samples. In order to achieve this goal, the present disclosure adopts the extracted multimodal fusion feature to calculate the relationship between the sample and the category to which the sample belongs. The use of this multimodal fusion feature enhanced prototype network generates more discriminative prototypes.

Firstly, the prototypes cₖ of the k class, the prototype c_{P}∈R^{d} of the labeled positive sample, and the prototype c_{U}∈R^{d} of the unlabeled sample are respectively calculated through the fusion feature, and the calculation method is as follows:

$${c}_{P} = \frac{1}{\vert {X}_{P} \vert} {\sum }_{{x}_{{n}_{p}} ∈ {X}_{P}} O ( {x}_{{n}_{p}} ) , {c}_{U} = \frac{1}{\vert {X}_{P} \vert} {\sum }_{{x}_{{n}_{U}} ∈ {x}_{U}} O ( {x}_{{n}_{U}} )$$

Where, xₙ_{ₚ} is the labeled positive sample, xₙ_{ᵤ} is the unlabeled sample, and O(xₙ_{ₚ}) and O(xₙ_{ᵤ}) are the labeled positive sample and unlabeled fusion features respectively.

Since most of the unlabeled samples belong to the negative class and only πₚ is the positive class, the prototype c_{U} of the unlabeled sample can be regarded as the prototype c_{N} of the biased negative class sample, and thus the sample label can be regarded as the pseudo label of Y=−1.

The Euclidean distance between each sample xᵢ and each prototype cₖ in the embedding space is then calculated to obtain the probability distribution p_{ϕ}(y=k|xᵢ) of the binary classification.

$${p}_{ϕ} ( y = k \vert {x}_{i} ) = \frac{exp ( - {‖ O ( {x}_{i} ) - {c}_{k} ‖}_{2}^{2} )}{{\sum }_{k} exp ( - {‖ O ( {x}_{i} ) - {c}_{k} ‖}_{2}^{2} )}$$

O(xᵢ) is the fused feature of the sample xᵢ, wherein k is the category, k is 2 in the present disclosure, R^{d} is the feature space, wherein d is the specific feature dimension, and y is the probability of being equal to a particular category k.

Finally, the loss function of all samples in the data set is calculated to train the multimodal feature enhanced prototype network by minimizing the negative logarithmic probability, and because c_{N} in the current prototype network is biased, the loss function of the multimodal feature enhanced prototype network is:

$${L}_{m p n} = \frac{1}{\vert X \vert} [ {‖ O ( {x}_{i} ) - {c}_{k} ‖}_{2}^{2} + log {\sum }_{k} exp ( - {‖ O ( {x}_{i} ) - {c}_{k} ‖}_{2}^{2} ) ] .$$

And further, in order to solve that problem that the multimodal feature enhanced prototype network has deviation due to unmarked sample, a contrastive learning network based on PU learning, namely the PU contrastive learn network, is introduced, so that not only the similarity among samples of the same class in the prototype network can be improved, the similarity among samples of different classes can be reduced, and more distinct feature representation of category can be obtained. At the same time, the unbiased PU learning strategy can solve the deviation caused by the positive sample data in the unlabeled sample prototype, and finally obtain more distinctive and representative features and prototype networks to improve the accuracy of the classification task.

Because the task of the present disclosure only has a part of positive sample labels and a large number of unlabeled samples, in order to solve the deviation, an unbiased PU learning strategy is adopted, which depends on an unbiased risk assessor, supposing that g_{ϕ} is an arbitrary decision function and lₚᵤ is a loss function, the empirical risk of the positive sample is fₚ⁺(g_{ϕ})=Eₓ_{ᵢ}_{∈X}_{_{P}}[lₚᵤ(g_{ϕ}), +1], the empirical risk of the negative sample is fₙ⁻(g_{ϕ})=Eₓ_{ᵢ}_{∈X}_{_{N}}[lₚᵤ(g_{ϕ}), −1], then the overall empirical risk of the function go on the traditional binary classification task with positive samples and negative samples is:

$$f ( {g}_{ϕ} ) = {π}_{P} {f}_{p}^{+} ( {g}_{ϕ} ) + {π}_{n} {f}_{n}^{-} ( {g}_{ϕ} )$$

Wherein, π_{P} is the class prior probability for the positive sample and πₙ is the class prior probability for the negative sample.

During the training of PU learning, since the distribution of data is obtained by sampling, and there are only positive samples and no negative samples, fₙ⁻(g_{ϕ}) needs to be obtained by approximation, and since πₙfₙ⁻(g_{ϕ})=fₙ⁻(g_{ϕ})−ϕₚfₚ⁻(g_{ϕ}), the risk assessment function of PU learning can be approximated as:

$${\hat{f}}_{P u} ( {g}_{ϕ} ) = {π}_{p} {\hat{f}}_{p}^{+} ( {g}_{ϕ} ) - {π}_{P} {\hat{f}}_{p}^{-} ( {g}_{ϕ} ) + {\hat{f}}_{u}^{-} ( {g}_{ϕ} ) ;$$

wherein, \hat{f}ₚ⁻(g_{ϕ}) and \hat{f}ₚ⁻(g_{ϕ}) are calculated as follows:

$${\hat{f}}_{p}^{-} ( {g}_{ϕ} ) = \frac{1}{\vert {X}_{P} \vert} {\sum }_{i = 1}^{{n}_{p}} {l}_{p u} ( {g}_{ϕ} ( {x}_{{n}_{p}} ) , - 1 )$$

$${\hat{f}}_{u}^{-} ( {g}_{ϕ} ) = \frac{1}{\vert {X}_{U} \vert} {\sum }_{i = 1}^{{n}_{u}} {l}_{p u} ( {g}_{ϕ} ( {x}_{{n}_{u}} ) , - 1 ) ;$$

According to the definition, πₙfₙ⁻(g_{ϕ}) should be non-negative, but when the PU is learning and training, the approximate value fₙ⁻(g_{ϕ})−πₚfₚ⁻(g_{ϕ}) may be negative in the later stage of learning, resulting in the phenomenon of overfitting. In order to solve the problem, the present disclosure introduces the Max operation into the risk loss function, and the specific formula is as follows:

$${\hat{f}}_{P u} ( {g}_{ϕ} ) = {π}_{p} {\hat{f}}_{p}^{+} ( {g}_{ϕ} ) + max \{ 0 , {\hat{f}}_{u}^{-} ( {g}_{ϕ} ) - {π}_{P} {\hat{f}}_{p}^{-} ( {g}_{ϕ} ) \} ;$$

As can be seen from the above formula, under the condition that π_{P} is known, in order to further reduce the risk loss function, it is necessary to reduce \hat{f}ᵤ⁻(g_{ϕ})−ϕ_{P}\hat{f}ₚ⁻(g_{ϕ}), that is, to accurately identify the positive sample from the unlabeled sample. In order to achieve the purpose, a contrastive learning method is introduced, and the feature representation in the unlabeled multimodal data is mined in a self-supervised manner by means of the contrastive learning.

Due to the key of contrastive learning lies in the selection of positive and negative sample pairs. In order to construct positive and negative samples sample pairs with differentiation, in this embodiment, the fusion feature Oᵢ obtained by training and the prototype cₖ are combined using a function φ(x):

zᵢ=φ(Oᵢ,cₖ);
wherein, φ(x) can be weighted average, splicing, taking the maximum value and other forms to obtain zᵢ∈R^{d}.

Specifically, Z_{P}ⁱ, Z_{P}ⁱ={z_{P}¹, Z_{P}², . . . , z_{P}ⁿ^{ₚ}}, is obtained by calculating the fused feature O_{P} of the positive sample label and the positive class prototype c_{P} as a positive sample pair, while Z_{U}^{j}, Z_{U}^{j}={z_{U}¹, z_{U}², . . . , z_{U}ⁿ^{ᵤ}}, is obtained by calculating the fused feature O_{U} of the unlabeled sample and the unlabeled prototype c_{U}. Since some samples in Z_{U}^{j} are negative classes, only π_{P} has a probability of being a positive sample, so it can be approximated as a biased negative sample prototype. In order to solve the problem of bias, a contrastive learning loss function L_{cpu} is constructed in combination with the above unbiased risk estimation method, and the PU contrastive learning network is optimized based on this function. The formula of the contrastive learning loss function L_{cpu} is as follows:

$${L}_{c p u}^{( i , j )} = - log \frac{exp ( {Z}_{P}^{i} \cdot {π}_{P} {Z}_{U}^{j} )}{\begin{matrix}{\sum }_{i ∈ \{ 1 , 2 , … {n}_{p} \} , j ∈ \{ 1 , 2 , … {n}_{u} \}} ( exp ( {Z}_{P}^{i} \cdot {π}_{P} {Z}_{U}^{j} ) + \\ exp ( max ( 1 {e}^{- 5} , {Z}_{P}^{i} \cdot ( 1 - {π}_{P} ) {Z}_{U}^{{j}^{′}} ) ) )\end{matrix}} ,$$

Due to \hat{f}ᵤ⁻(g_{ϕ})−π_{P}\hat{f}ₚ⁻(g_{ϕ})≥0 in the above, max(1e⁻⁵, Z_{P}ⁱ·(1−π_{P})Z_{U}^{j′}) needs to be calculated in the formula.

S3, inputting the multimodal data into a trained unbiased classification model, and outputting the category to which the multimodal data belongs. The specific operation steps are described as follows:
(1) input the collected EEG multimodal data and text multimodal data into the feature extraction and fusion network to generate a comprehensive and representative fusion feature Oᵢ;
(2) calculate the Euclidean distance between the fusion feature Oᵢ and each prototype cₖ in the multimodal prototype network in the embedding space to obtain the probability distribution p_{ϕ} of sample classification; and
(3) deciding the category to which the multimodal data belongs according to the probability distribution p_{ϕ}.

It can be seen that after the training of the model is completed, the model can accurately determine its category only by using the obtained multimodal (EEG, text) data as input in the reasoning stage, so as to complete the classification task in the environment of unbalanced data and limited labels.

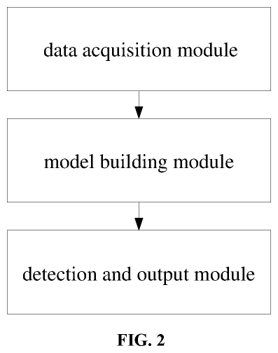

On the other hand, an anomaly detection system based on PU contrastive learning within a multimodal prototype network is proposed by the present disclosure, as shown in the FIG. 2, which is used to implement the aforementioned the anomaly detection system based on PU contrastive learning within a multimodal prototype network, including:
a data acquisition module, which is configured to acquire multimodal data, wherein the multimodal data includes EEG modal data and text modal data;
a model building module, which is configured to construct and train an unbiased classification model; the unbiased classification model includes a feature extraction and fusion network, a multimodal feature enhanced prototype network and a PU contrastive learning network; the feature extraction and fusion network generates a fusion feature according to the multimodal data, the fusion feature is clustered by multimodal feature enhanced prototype network. Due to the lack of labeled negative samples and imbalanced data, there is a certain degree of deviation in the clustering results. To solve this problem, a PU contrastive learning network is used to estimate the unbiased risk of the biased clustering results mentioned above, correct the resulting deviation, and classify multimodal data; and
a detection and output module, which is configured to input the multimodal data into a trained unbiased classification model, and output the category to which the multimodal data belongs.

Various embodiments of the present specification are described in a progressive manner, and each embodiment focuses on the description that is different from the other embodiments, and the same or similar parts between the various embodiments are referred to with each other. For the device disclosed in the embodiment, since it corresponds to the method disclosed in the embodiment, the description is relatively simple, and the correlation is described with reference to the method part.

The above description of the disclosed embodiments enables those skilled in the art to implement or use the present disclosure. Various amendments to the embodiments will be apparent to those skilled in the art. The general principles defined herein may be implemented in other embodiments without departing from the spirit or scope of the disclosure. Therefore, the present disclosure will not be limited to the embodiments shown herein but is to be accorded the widest scope consistent with the principles and novel features disclosed herein.

1. An anomaly detection method based on positive unlabeled (PU) contrastive learning within a multimodal prototype network, comprising:
acquiring multimodal data, wherein the multimodal data comprises electroencephalography (EEG) modal data and text modal data;
constructing and training an unbiased classification model; wherein the unbiased classification model comprises a feature extraction and fusion network, a multimodal feature enhanced prototype network and a PU contrastive learning network; the feature extraction and fusion network generates a fusion feature according to the multimodal data, and the fusion feature is clustered by the multimodal feature enhanced prototype network to obtain a biased cluster result; and finally, carrying out unbiased risk estimation on the biased cluster result by using the PU contrastive learning network and correcting a deviation to realize a classification of the multimodal data;
wherein, the fusion feature is merged with a prototype cₖ through the PU contrastive learning network to obtain a sample pair Zᵢ; and an unbiased risk estimation function is constructed based on the sample pair Zᵢ, wherein the unbiased risk estimation function is a PU contrastive learning loss function; the PU contrastive learning network is optimized according to the PU contrastive learning loss function;
wherein an operation of merging the fusion feature with the prototype cₖ to obtain the sample pair Zi comprises:
calculating a positive sample pair Z_{P}ⁱ, Z_{P}ⁱ={z_{P}¹, Z_{P}², . . . , z_{P}ⁿ^{ₚ}}, by a fusion feature O_{P} of a partial labeled positive sample X_{P} and a positive class prototype c_{P}, and calculating a sample pair Z_{U}^{j}, Z_{U}^{j}={z_{U}¹, z_{U}², . . . , z_{U}ⁿ^{ᵤ}}, by a fusion feature O_{U} of an unlabeled sample X_{U} and an unlabeled prototype c_{U};
inputting the multimodal data into a trained unbiased classification model, and outputting a category to which the multimodal data belongs, comprising:
inputting the multimodal data into the feature extraction and fusion network to generate a fusion feature Oᵢ;
calculating an Euclidean distance between the fusion feature Oᵢ and each prototype cₖ in the multimodal feature enhanced prototype network in an embedding space to obtain a probability distribution p_{ϕ} of a sample classification; and
deciding the category to which the multimodal data belongs according to the probability distribution p_{ϕ}.

2. The anomaly detection method based on the PU contrastive learning within the multimodal prototype network according to claim 1, wherein the acquiring the multimodal data comprises preprocessing the multimodal data;
dividing the multimodal data into the partial labeled positive sample X_{P}, X_{P}={x₁, x₂, . . . , xₙ_{ₚ}}, and the unlabeled sample X_{U}, X_{U}={x₁, x₂, . . . , xₙ_{ᵤ}}, wherein a label of the partial labeled positive sample X_{P} is Y=+1, and the unlabeled sample X_{U} has no label.

3. The anomaly detection method based on the PU contrastive learning within the multimodal prototype network according to claim 1, wherein the feature extraction and fusion network comprises a dilated convolutional network, a Bidirectional Encoder Representations from Transformers (BERT) model, and a multi-head self-attention mechanism;
the dilated convolutional network and the BERT model are respectively configured to extract features of the EEG modal data and the text modal data, and the multi-head self-attention mechanism is configured to fuse features of the multimodal data to generate the fusion feature.

4. The anomaly detection method based on the PU contrastive learning within the multimodal prototype network according to claim 3, wherein the multimodal prototype network calculates k-class prototypes cₖ by fusing features, cₖ={c_{P}, c_{U}}, wherein c_{P} is a prototype of the partial labeled positive sample X_{P}, wherein the prototype of the partial labeled positive sample X_{P} is the positive class prototype, c_{P}∈R^{d}; c_{U} is a prototype of the unlabeled sample X_{U}, wherein the prototype of the unlabeled sample X_{U} is the unlabeled prototype c_{U}∈R^{d}; and
calculating the Euclidean distance between each sample xᵢ and each prototype cₖ in the embedding space to obtain the probability distribution p_{ϕ}(y=k|xᵢ) of a binary classification;
wherein, k is the category, y is a probability of being equal to a particular category k, R^{d} is a feature space, and d is a feature dimension.

5. The anomaly detection method based on the PU contrastive learning within the multimodal prototype network according to claim 4, wherein a loss function of the multimodal feature enhanced prototype network is:
$${L}_{m p n} = \frac{1}{\vert X \vert} [ {‖ O ( {x}_{i} ) - {c}_{k} ‖}_{2}^{2} + log {\sum }_{k} exp ( - {‖ O ( {x}_{i} ) - {c}_{k} ‖}_{2}^{2} ) ] ;$$
wherein, X is a data sample, and O(xᵢ) is a fusion feature of a sample xᵢ.

6. The anomaly detection method based on the PU contrastive learning within the multimodal prototype network according to claim 1, wherein the PU contrastive learning loss function is:
$${L}_{c p u}^{( i , j )} = - log \frac{exp ( {Z}_{P}^{i} \cdot {π}_{P} {Z}_{U}^{j} )}{\begin{matrix}{\sum }_{i ∈ \{ 1 , 2 , … {n}_{p} \} , j ∈ \{ 1 , 2 , … {n}_{u} \}} ( exp ( {Z}_{P}^{i} \cdot {π}_{P} {Z}_{U}^{j} ) + \\ exp ( max ( 1 {e}^{- 5} , {Z}_{P}^{i} \cdot ( 1 - {π}_{P} ) {Z}_{U}^{{j}^{′}} ) ) )\end{matrix}}$$
wherein, πₚ is a prior probability of a positive sample.

7. An anomaly detection system based on PU contrastive learning within a multimodal prototype network, comprising:
a data acquisition module, configured to acquire multimodal data, wherein the multimodal data comprises EEG modal data and text modal data;
a model building module, configured to construct and train an unbiased classification model; wherein the unbiased classification model comprises a feature extraction and fusion network, a multimodal feature enhanced prototype network and a PU contrastive learning network; the feature extraction and fusion network generates a fusion feature according to the multimodal data, and the fusion feature is clustered by the multimodal feature enhanced prototype network to obtain a biased cluster result; and finally, carrying out unbiased risk estimation on the biased cluster result by using the PU contrastive learning network and correcting a deviation to realize a classification of the multimodal data;
wherein, the fusion feature is merged with a prototype cₖ through the PU contrastive learning network to obtain a sample pair Zᵢ; and an unbiased risk estimation function is constructed based on the sample pair Zᵢ, wherein the unbiased risk estimation function is a PU contrastive learning loss function; the PU contrastive learning network is optimized according to the PU contrastive learning loss function;
wherein an operation of merging the fusion feature with the prototype cₖ to obtain the sample pair Zᵢ comprises:
calculating a positive sample pair Z_{P}ⁱ, Z_{P}ⁱ={z_{P}¹, Z_{P}², . . . , z_{P}ⁿ^{ₚ}}, by a fusion feature O_{P} of a partial labeled positive sample X_{P} and a positive class prototype c_{P}, and calculating a sample pair Z_{U}^{j}, Z_{U}^{j}={z_{U}¹, z_{U}², . . . , z_{U}ⁿ^{ᵤ}}, by a fusion feature O_{U} of an unlabeled sample X_{U} and an unlabeled prototype c_{U};
a detection and output module, configured to input the multimodal data into a trained unbiased classification model, and output a category to which the multimodal data belongs, comprising:
inputting the multimodal data into the feature extraction and fusion network to generate a fusion feature Oᵢ;
calculating an Euclidean distance between the fusion feature Oᵢ and each prototype cₖ in the multimodal feature enhanced prototype network in an embedding space to obtain a probability distribution p_{ϕ} of a sample classification; and
deciding the category to which the multimodal data belongs according to the probability distribution p_{ϕ}.
